(19) United States
(12) Plant Patent Application Publication
(10) Pub. No.: US 2021/0368661 P1
(43) Pub. Date: Nov. 25, 2021

(54) Olive tree named 'I-20'

(50) ;

(71) Applicant: Todolivo S.L., Córdoba (ES)

(72) Inventors: Jose María GÓMEZ PORRAS, Córdoba (ES); Luis RALLO ROMERO, Córdoba (ES); Diego BARRANCO NAVERO, Córdoba (ES); Concepción MUÑOZ DÍEZ, Córdoba (ES); Carlos TRAPERO RAMÍREZ, Córdoba (ES); Pedro VALVERDE CABALLERO, Córdoba (ES)

(73) Assignee: Todolivo S.L., Córdoba (ES)

(21) Appl. No.: 17/326,877

(22) Filed: May 21, 2021

(30) Foreign Application Priority Data
May 21, 2020 (ES) 20205550

(51) Int. Cl. A01H 6/00

(52) U.S. Cl. PLT/158

(57) ABSTRACT

A new and distinct variety of olive tree, herein referred to by its cultivar name, ‘1-20’, is provided which forms a dropping growth habit. Fruit with a low weight and obtuse shape is produced. The fruit exhibit dark violet coloration and the fruit nipple is absent or weak. The stone of the fruit of the new variety is of low weight, moderately elongated and possess a weakly rugose surface.

Botanical/commercial classification: Latin name—Olea europaea L. Common name—Olive tree. Varietal denomination—‘1-20’.

### CROSS-REFERENCE TO RELATED APPLICATIONS

This application claims priority to Spanish Application No. 20205550, which was filed at Spanish Office of Vegetable Varieties in Spain on May 21, 2020, the contents of which are hereby incorporated by reference for all purposes.

### SUMMARY OF THE INVENTION

The new variety of olive tree of the present invention was created by a controlled cross in Spain wherein two parents which previously had been studied were crossed in the hope that they would contribute the desired characteristics. The female parent (i.e., seed parent) of the new variety was the ‘ARBOSANA’ variety (unpatented). The male parent (i.e., pollen parent) was the ‘KORONEIKI’ variety (unpatented).

The parentage can be summarized as follows:
‘ARBOSANA’ x ‘KORONEIKI’.

The seeds resulting from the above pollination were sown and small plants were obtained which were physically and biologically different from each other. Selective study resulted in the identification of a single plant of the new variety.

The new variety's color is a less intense green when compared to the color of the ‘Arbosana’ variety. The new variety's size is smaller than the ‘Koroneiki’ variety. A non-parental variety that the new variety is most similar to is the ‘Arbequino variety. The new variety's fat yield is similar to that of ‘Arbequino’ but differs from ‘Arbequino’ in that it possesses a very high and constant productivity.

It was found that the new variety of the present invention possesses the following combination of characteristics:
(a) the tree has a drooping growth habit
(b) the fruit weight is low and the fruit color is dark violet
(c) the fruit is obtuse and weakly asymmetric
(d) the fruit either possesses a weak or absent nipple
(e) the stone is moderately elongated with a low weight and weakly rugose surface.

The new variety well meets the needs of the horticultural industry and has not been commercialized. The new variety has an easy agronomic management.

The new variety can be readily distinguished from related similar varieties. The new variety has a small variety size.

The new variety has been found to undergo asexual propagation in Spain by vegetative cuttings. Asexual propagation by vegetative cutting in Spain has shown that the characteristics of the new variety are stable and are strictly transmissible by such asexual propagation from one generation to another. Accordingly, the new variety undergoes asexual propagation in a true-to-type manner.

The new variety has been named ‘1-20’.

### BRIEF DESCRIPTION OF THE PHOTOGRAPHS

The accompanying photographs show as nearly true as it is reasonably possible to make the same, in a color illustration of this character, typical specimens of the plant parts of the new variety.

FIG. 1 illustrates the growth of the new variety.

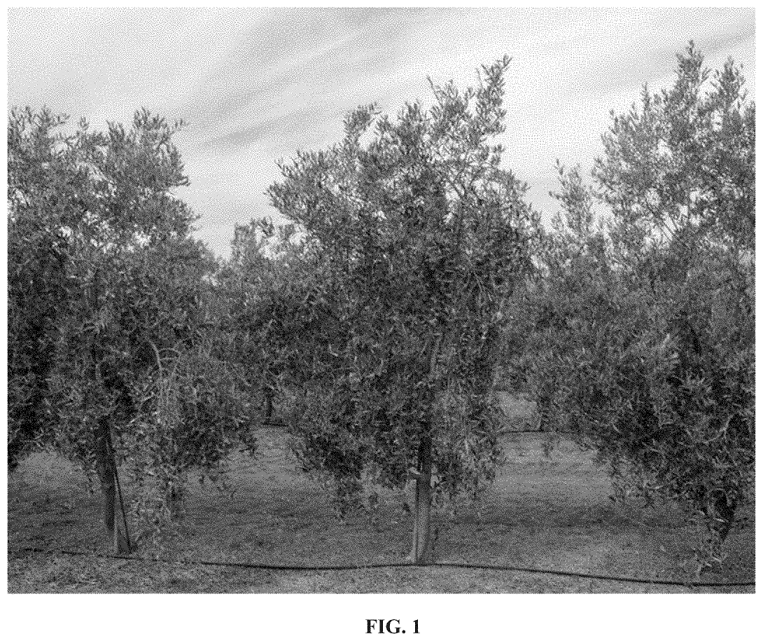

FIG. 2 illustrates the foliage of the new variety.

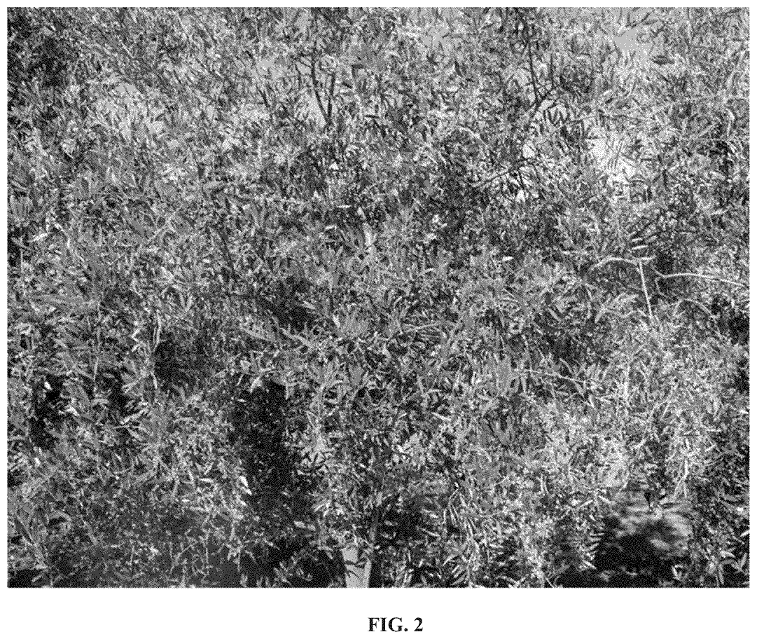

FIG. 3 illustrates specimens of the new variety's fruit.

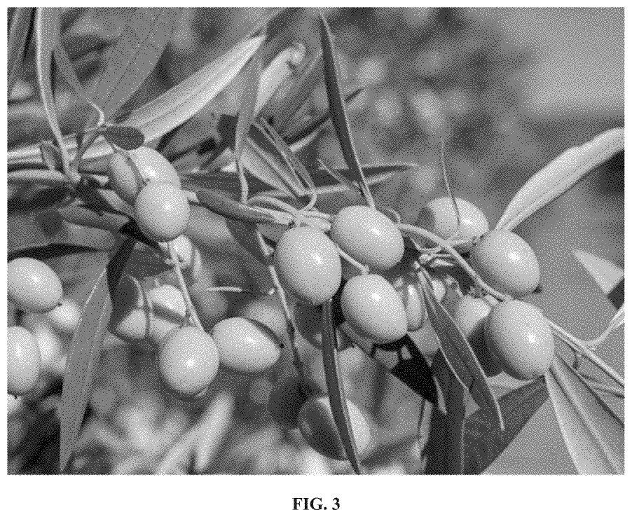

### DETAILED BOTANICAL DESCRIPTION

The chart used in the identification of the colors is that of The Royal Horticultural Society (R.H.S. Colour Chart, 2007 edition), London, England. The terminology which precedes reference to the chart has been added to indicate the corresponding color in more common terms. The description is based on data collected from a four-year-old specimen during 2019 in Villafranca (Cordoba, Spain).
Plant:
   Growth habit.—erect and weeping.
Fruit:
   Average weight.—low.
   Shape-apex color designation.—obtuse.
   (Flesh color).—dark violet.
   Ripening.—medium.
Stone:
   Quantity.—obtuse.
   Shape.—moderately elongated.
   Average weight.—Low.
   Texture.—Weakly rugose.
   Mucron.—Present.
Development:
   Productivity.—very high and constant.
   Plant/fruit disease, pest resistance.—Resistant to Cucumber mosaic virus (CMV), Cherry leaf roll virus (CLRV), Strawberry latent ringspot virus (SLRSV), Arabis mosaic virus (ArMV) and Repilo. Tolerant to Verticilium and tuberculosis.

The new ‘1-20’ variety has not been observed under all possible environmental conditions to date. Accordingly, it is possible that the phenotypic expression may vary somewhat with changes in light intensity and duration, cultural practices, and other environmental conditions.

We claim:

1. A new and distinct variety of olive tree characterized by the following combination of characteristics:
(a) a tree with a drooping growth habit,
(b) a fruit with low weight and a dark violet color,
(c) the fruit is obtuse and weakly asymmetric,
(d) the fruit either possesses a weak or absent nipple, and
(e) a stone that is moderately elongated with a low weight and a weakly rugose surface; substantially as herein shown and described.
